(19) United States
(12) Patent Application Publication
(10) Pub. No.: US 2007/0020244 A1
(43) Pub. Date: Jan. 25, 2007

(54) Fiber constructs and process of fiber fabrication

(75) Inventors: Andrew C. Aun Wan, Singapore (SG); Kam W. Leong, Ellicott City, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/396,374

(22) Filed: Mar. 30, 2006

Related U.S. Application Data
(60) Provisional application No. 60/667,107, filed on Mar. 30, 2005.

(51) Int. Cl. A61K 39/12; A61K 48/00; A61K 38/16; A61K 35/12; A61K 9/70

(52) U.S. Cl. 424/93.7; 424/443; 424/204.1; 514/2; 514/44; 977/906

(57) ABSTRACT

Described herein are fiber compositions, methods of generating the fiber compositions, and methods of using the fiber compositions in various applications utilizing fiber constructs, including for example, tissue engineering.

The present application claims the benefit of U.S. provisional application 60/667107, filed Mar. 30, 2005, which is incorporated herein by reference in its entirety.

### BACKGROUND OF THE INVENTION

In the emerging field of tissue engineering, biodegradable polymeric scaffolds are used as templates for tissue regeneration. Science 260, 920-926 (1993). Cells attach to these scaffolds, proliferate, differentiate if necessary, and develop into tissue-like materials to replace or augment the damaged tissue functions. So far, most of these scaffolds play a mainly structural role of supporting cell adhesion and defining the framework of tissue growth. However, optimal tissue engineering requires more than an inert scaffold that serves merely as a substrate for cell attachment and growth. Optimal tissue engineering demands that cues or signal molecules in the form of adhesion molecules, growth and differentiation factors, or even plasmid DNA be incorporated into these scaffolds in a spatially defined manner to orchestrate the growth of new tissue. Science 295, 1014-1017 (2002); Nature Biotech. 19, 934-939 (2001). While current bioengineering technologies may realize this objective to various degrees of success, precise control of spatial distribution and delivery of these signal molecules may be better achieved if the basic structural unit of the scaffold is itself endowed with the molecules. Particularly, this could be improved if the biological factors are incorporated in fibers, which can be then fabricated by textile engineering techniques into scaffolds of controlled porous architecture. While cells and their contingent extracellular matrix (ECM) and proteins make up the basic biological unit, fibers comprise the basic structural unit, and a combination of both would form the “biostructural unit” that could be translated to a higher level of organization. Described herein are methods showing how proteins, conjugated ligands and cells can be integrated with a structural fiber component to form a biostructural unit, and associated benefits of such architectures.

To realize the concept of a biostructural unit, a process is described that allows encapsulation of biological materials at ambient temperature and under aqueous conditions. The process of interfacial polyelectrolyte complexation (IPC) is a process of self-assembly that occurs when two oppositely charged polyelectrolytes come together. Recently, it has garnered considerable interest as an attractive method for the controlled assembly of multicomponent nanostructures. Science 277 1232 (1997). Polyelectrolyte multilayer assemblies have been used with promising results for application as anti-reflection coatings, light-emitting diodes and microcapsules. See, e.g., Nature Materials 1 59-63 (2002); Supramolecular Science 4 67-73 (1997); Langmuir 17 2036-2042 (2001); Adv. Mater. 13 1324-1327 (2001). Fibers have also been created by means of a similar self assembly process, although no mechanism of fiber formation has been offered to date. J. Appl. Polym. Sci. 79, 437-446 (2001).; Macromol. Rapid Commun. 23, 540-543 (2002). Here is now described a mechanism of fiber formation via interfacial polyelectrolyte complexation, and how this mechanism allows the unique encapsulation characteristics required for the formation of the biostructural units.

### SUMMARY OF THE INVENTION

Described herein are fiber compositions, methods of generating the fiber compositions, and methods of using the fiber compositions in various applications utilizing fiber constructs, including for example, tissue engineering.

One aspect is a fiber including at least two polyionic fiberelles and at least one bioactive material encapsulated within the fiber. Other aspects are those wherein the polyanionic fiberelles are composed of at least one polycationic polymer and at least one polyanionic polymer; those wherein the polycationic polymer is biodegradable or biocompatible; those wherein the polycationic polymer is selected from the group consisting of natural and synthetic carbohydrate or polypeptide polymers having a net positive chare; those wherein the polycationic polymer is selected from the group consisting of chitin, chitosan, poly(lysine), and combinations thereof; those wherein the polyanionic polymer is biodegradable or biocompatible; those wherein the polyanionic polymer is selected from the group consisting natural and synthetic carbohydrate or polypeptide polymers having a net negative charge; those wherein the polyanionic polymer is selected from the group consisting of alginate, gellan, polyacrylic acid, and combinations thereof; those wherein the polycationic polymer is chitin or chitosan, and the polyanionic polymer is selected from the group consisting of alginate, gellan, or a combination thereof; those wherein the polycationic polymer is chitin and the polyanionic polymer is alginate; those wherein the bioactive material is selected from drugs, proteins, DNA, RNA, cells, viruses, microparticles, nanoparticles, contrast agents, or combinations thereof; those wherein the bioactive material provides at least one extracellular matrix molecule suitable for stabilizing cells; those wherein the extracellular matrix molecule is a protein or drug capable of recapitulating an extracellular matrix of natural tissue; those wherein the bioactive material is cells and the fiber provides an anchoring site for cellular products secreted from the cells; those wherein the fiber comprises between two and 10,000 fibrelle (including any range that has upper and lower limits between 2 and 10,000) those wherein the fiber comprises between 10 and 1000 (including any range that has upper and lower limits between 10 and 1,000) fibrelles; those wherein the fiber has a substantially homogenous cross-section; those wherein the cross-section is between about 0.5 and about 10 μm (including any range that has upper and lower limits between 0.5 and 10 μm); those wherein the fiber comprises domains of narrow cross-section interposed with beads having a larger cross-section; and those wherein the bead has a cross-section between about 2 and about 25 (including any range that has upper and lower limits between 2 and 25) times the cross-section of the narrower domains.

Another aspect is an article of manufacture including at least one fiber of any of the aforementioned embodiments. Other aspects include the article of manufacture: wherein the article is selected from braids, woven and non-woven fabrics, mesh, and combinations thereof.

Another aspect is a tissue engineering scaffold including at least one fiber comprising at least two polyionic fiberelles and at least one bioactive material encapsulated within the fiber. Other aspects include the tissue engineering scaffold wherein the bioactive material is selected from drugs, proteins, DNA, RNA, cells, viruses, microparticles, nanoparticles, contrast agents, or combinations thereof; those wherein the bioactive material provides an extracellular matrix suitable for stabilizing cells; those wherein the bioactive material is a protein or drug associated with tissue regeneration or imaging.

Another aspect is a method of preparing a fiber comprising at least two polyionic fiberelles and at least one bioactive material encapsulated within the fiber, the method including the steps of:
(a) providing an aqueous solution of a polyanionic polymer and an aqueous solution of a polycationic polymer, wherein at least one of the polyanionic polymer solution or the polycationic polymer solution further comprises at least one bioactive material;
(b) contacting the polyanionic polymer solution and the polycationic polymer solution under conditions conducive to form an interface; and
(c) pulling a fiber from the interface.

Other aspects of the method are those, wherein the interface between the polyanionic polymer solution and the polycationic polymer solution has a cross-section of less than about 10 mm²; those wherein the cross-section of the interface is between about 1 mm² and about 5 (including any range that has upper and lower limits between 1 and 5) mm²; those wherein the fiberelles are composed of at least one polycationic polymer and at least one polyanionic polymer; those wherein the polycationic polymer is biodegradable or biocompatible; those wherein the polyanionic polymer solution has a polyanionic polymer concentration of less than about 10% (w/v); those wherein the polycationic polymer is selected from the group consisting of natural and synthetic carbohydrate or polypeptide polymers having a net positive charge; those wherein the polycationic polymer is selected from the group consisting of chitin, chitosan, poly(lysine), and combinations thereof; those wherein the polyanionic polymer is biodegradable or biocompatible; those wherein the polyanionic polymer is selected from the group consisting natural and synthetic carbohydrate or polypeptide polymers having a net negative charge; those wherein the polyanionic polymer is selected from the group consisting of alginate, gellan, polyacrylic acid, and combinations thereof; those wherein the polycationic polymer is chitin or chitosan, and the polyanionic polymer is selected from the group consisting of alginate, gellan, or a combination thereof; those wherein the polycationic polymer is chitin and the polyanionic polymer is alginate; those wherein the bioactive material is selected from drugs, proteins, DNA, RNA, cells, viruses, microparticles, nanoparticles, contrast agents, collagen, or combinations thereof; those wherein the bioactive material provides at least one extracellular matrix molecule suitable for stabilizing cells; those wherein the extracellular matrix molecule is a protein or drug capable of recapitulating an extracellular matrix of natural tissue; those wherein the bioactive material is cells and the fiber provides an anchoring site for cellular produces secreted from the cells; those wherein the pulling of the fiber from the interface comprises a substantially continuous linear pulling motion; those wherein the fiber is pulled at a rate of between about 0.1 mm/second and about 100 mm/second (including any range that has upper and lower limits between 0.1 and 100); those wherein the pulling force is generated by connecting the fiber to a circular winding means and rotating the winding means at the pull rate; those wherein the fiber comprises between two and 10,000 (including any range that has upper and lower limits between 2 and 10,000) fibrelles; those wherein the fiber comprises between 10 and 1000 (including any range that has upper and lower limits between 10 and 1,000) fibrelles; those wherein the fiber has a substantially homogenous diameter; those wherein the diameter is between about 0.1 μm and about 20 (including any range that has upper and lower limits between 0.1 and 20) μm; those wherein the diameter is between about 1 μm and about 5 (including any range that has upper and lower limits between 1 and 5) μm; those wherein the step of pulling the fiber from the interface is done at a rate less than the rate at which beading occurs; those wherein the fiber comprises domains of narrow diameter interposed with beads having a larger diameter; those wherein the step of pulling the fiber from the interface is done at a rate sufficient to induce beading; or those wherein the bead has a diameter of between about 2 and about 25 (including any range that has upper and lower limits between 2 and 25) times the diameter of the narrower domains.

Another aspect is a method of tissue engineering including the steps of:
(a) providing at least one fiber comprising at least two polyionic fiberelles and at least one bioactive material encapsulated within the fiber, or an article of manufacture composed of said fiber;
(b) shaping the fiber or article of manufacture into a two- or three-dimensional scaffold suitable for growth of the engineered tissue;
(c) contacting the scaffold with cells, growth factors, proteins, drugs, DNA, RNA, or combinations thereof under conditions conducive to tissue growth.

Another aspect is a product made by any of the processes delineated herein. In another aspect, the method is that wherein the bioactive material is selected from drugs, proteins, DNA, RNA, cells, viruses, microparticles, nanoparticles, contrast agents, collagen or combinations thereof.

Another aspect is a method of treating a subject including administering a composition (e.g., a fiber, fiber complex, scaffold, tissue, etc.) herein to the subject.

Another aspect is a method of generating tissue in a subject including administering a composition (e.g., a fiber, fiber complex, scaffold, tissue, etc.) herein to the subject.

Another aspect is a fiber herein, wherein each fibrelle is formed by interfacial polyelectrolyte complexation. Other aspects are those wherein each fiberelle is formed by interfacial complexation of at least one polycationic polymer and at least one polyanionic polymer; those wherein at least one polycationic polymer or at least one polyanionic polymer is biodegradable or biocompatible.

Another aspect is a fiber composition delineated herein including a therapeutic agent and optionally, a pharmaceutically acceptable carrier. The composition can also include an additional therapeutic agent (e.g., antibiotics (e.g., penicillin, streptomycin), growth factors (e.g., human growth factor), proteins, cell proliferation agents, etc.).

One aspect is a method of treating or ameliorating a subject suffering from or susceptible to a disease or disorder, or symptom thereof. The method includes the step of administering to the subject a composition herein sufficient to treat or ameliorate the disease or disorder or symptom thereof under conditions such that the disease or disorder or symptom thereof is treated. In certain preferred embodiments, the subject is a human. In certain preferred embodiments, the subject is a subject identified as being in need of such treatment. In certain preferred embodiments, the step of administering comprises administering the fiber composition topically or intramuscularly.

In another aspect, an embodiment provides kits for producing the fiber compositions herein. In one embodiment, the kit includes an effective amount of polyanionic polymer, polycationic polymer, a bioactive material, and instructions for forming a fiber from an interface of solutions of the two polymers.

Another aspect is a method of making a fiber composition of any of the formulae herein, comprising taking a precursor compound (or intermediate) and reacting it with one or more chemical reagents to provide the compound of the formulae herein. The method can include one or more of the synthetic steps specifically delineated herein. Accordingly, another aspect is a compound made by a process delineated herein. The process can include one or more steps, reagents and starting materials as delineated herein using chemical reactions, techniques and protocols as delineated herein.

In certain embodiments, each polyionic (e.g., polyanionic, polycationic) polymer solution independently has a concentration that is any number between about 0 and 500 (inclusive) mg/mL; or about <10% (w/v) (e.g., any number between about 0 and 10%, inclusive; <1%; between 0 and 1% inclusive, between 0.1 and 0.99% inclusive). In certain embodiments, the ratio of one polyionic polymer solution to another is any ratio wherein the numerator is any number between about 0 and 100 inclusive and the denominator is any number between 1 and 100 inclusive (e.g., 1:1; 1:2, 1:2.5; 1:4, 1:5, 1:10, 1:20, 1:50, 1:100, etc.).

Fiber complexes that are representative embodiments of the formulae herein and are useful in the methods are delineated herein.

### BRIEF DESCRIPTION OF THE DRAWINGS

FIG. 1: Scanning electron micrograph (SEM) of WSC-A fiber; (A) Fiber comprises of bead and fiber regions. Higher magnification of (B) fiber region; (C) bead region.

FIG. 2: The protein-fiber biostructural unit:

(A) Light micrograph of FITC-BSA encapsulated in WSC-A fiber; (B) Release profile of BSA from WSC-A fiber using different WSC to alginate concentration ratios: ◯0.33; ●0.4; □0.5; ⋄0.67; ♦1.0; (C) Percentage of differentiated PC12 cells at time points when supernatant containing released nerve growth factor (NGF) was added; loading concentration of NGF: Δ 66 ng/mL; □ 86 ng/mL; ♦ 112 ng/mL; ◯ 0 ng/mL (control).; (D) Confocal micrograph of biotinylated NGF immobilized on biotinylated fibers via avidin bridge, immunofluorescent staining with TRITC labelled antibody.

FIG. 3 The cell-fiber biostructural unit: (A) HDF at low density in bead region (light micrograph); cells are in different planes of focus, reflecting the 3-dimensional nature of encapsulation; (B) BPAEC in fiber region (confocal micrograph). The green and red labels indicate viable and non-viable cells respectively; Inset: light microscope image-arrows denote the change in the diameter of the fiber due to the presence of a BPAEC clump; (C) hMSC encapsulated in fiber, at 0 hours (light micrograph); (D) Clumps of hMSC have formed, at 24 hours.

FIG. 4 Assembly of hMSC biostructural units into a construct: (A) Apparatus for laying down the wet fiber containing hMSC; (B) Scaffold with encapsulated hMSC in tissue culture medium; (C) At higher magnification, after 6 weeks in vitro.

FIG. 5 illustrates mRNA expression of MSC control (lane 1), chondrogenic (lane 2) and osteogenic (lane 3) samples; (B) Von Kossa, and (C) Alizarin Red S histological staining of osteogenic sample.

FIG. 6 illustrates (a) Diagrammatic representation of the biostructural unit; (b) Construction of the biostructural unit: 1. creation of a kinetically frozen interface at the junction between two polyelectrolyte solutions; 2. disruption of the interface by the drawing process leads to scattered nucleation sites; 3. growth of “nuclear” fibres, accompanied by decrease in viscosity of surrounding matrix; 4. coalescence of “nuclear” fibres, leading to entrapment of cells and the formation of gel droplets (beads) along the fibre axis.; (c) Individual “nuclear” fibres spread out in the bead region of a nascent fibre that was immediately adhered upon a glass slide. (Scale bar=10 μm); (d) Dependence of critical draw rates on the concentration of polycation (chitosan in 0.15 M acetic acid): ⋄ fibre point; □ bead point.

FIG. 7 illustrates the process of fiber formation by interfacial polyelectrolyte complexation-continuous fiber production can be achieved by means of a roll-up apparatus.

FIG. 8: (A) Light microscope image of several air-dried fiber strands; (B) Fiber surface, composed of parallel ridges; (C) Individual “nuclear” fibers spread out in the bead region of a nascent fiber that was immediately adhered upon a glass slide. (Scale bar=10 μm); (D) Confocal micrograph of fiber composed of water soluble chitin and alginate labeled with quantum dots.

FIG. 9 illustrates light microscope stills of the fiber drawing process; N: nascent fiber; S: source; FA: fibrillar aggregate.

FIG. 10 illustrates turbidity curves from the contact of chitosan and alginate solutions; (chitosan, alginate concentrations in % w/v): + (0.125, 0.5); − (0.25, 0.5); — (0.5, 0.5); ♦ (0.125, 0.125); ● (0.25, 0.125); ▴ (0.5, 0.125); ⋄ (0.125, 0.25); ◯ (0.25, 0.25); Δ (0.5, 0.25).

FIG. 11 illustrates the dependence of critical draw rates on the concentration of chitosan in 0.15 M acetic acid: ⋄ fiber point; □ bead point.

FIG. 12 shows a diagrammatic representation of the interface between two polyelectrolyte solutions.

FIG. 13: Steps in the hypothesized mechanism of fiber formation by interfacial polyelectrolyte complexation (fiber cross-sectional area): 1. creation of a polyionic complex film at the junction between two polyelectrolyte solutions; 2. disruption of the interface by the drawing process leads to scattered domains of complexation that act as fiber nucleation sites; 3. growth of “nuclear” fibers, accompanied by decrease in viscosity of surrounding matrix; 4. coalescence of “nuclear” fibers, leading to the formation of gel droplets (beads) along the fiber axis.

FIG. 14 illustrates light microscope images of silica gel encapsulated in WSC-A fibers; Silica gel was dispersed in alginate solution at concentrations of (a) 0; (b) 10; (c) 30; (d) 50; (e) 100, and (f) 150 mg/mL. All photos are at the same magnification.

### DETAILED DESCRIPTION OF THE INVENTION

The use of biostructural units for tissue engineering can be seen at two levels. Firstly, it provides a method for the construction of scaffolds that present growth factors and other ligands in a well-defined manner to guide tissue regeneration, from the proliferation and differentiation of cells seeded in vitro or recruited from the host. At this level, the biostructural unit would take the form of a fibre, modified with encapsulated and/or immobilized molecules.

At another level, it can be seen as a method of creating tissue constructs with defined arrangements of one or more cell types. Such biostructural units would take the form of fibres containing encapsulated cells.

To date, the proposed concept of bioactive scaffold or tissue construct assembly via the assembly of biostructural units has been limited or difficult. Most polymer processes, specifically fibre fabrication, involve the use of heat, denaturing organic solvents, extreme pH or toxic crosslinking agents, all of which are detrimental to the incorporation of a biological component. These conditions are avoided in interfacial polyelectrolyte complexation, charge neutralization being the major driving force for the insolubilization of two water soluble polymers to form a fiber.

The reactants in interfacial polyelectrolyte complexation are both dissolved in an aqueous phase. In order that the complexation remains an interfacial phenomenon, mass transfer between the two phases must be reduced, so that free mixing between the two phases is avoided. This condition is satisfied by the formation of a polyelectrolyte complex “film” at the interface that limits exchange of the polyelectrolytes. Here, a “frozen structure” exists where homogenous complexation is prevented by a kinetic constraint. When materials are encapsulated in the fiber, this same viscous barrier prevents molecules or particles from diffusing into the other polyelectrolyte phase. This becomes important in the case of encapsulation of charged molecules or cells, where diffusion into the oppositely charged phase may result in precipitation and subsequent failure to draw fiber.

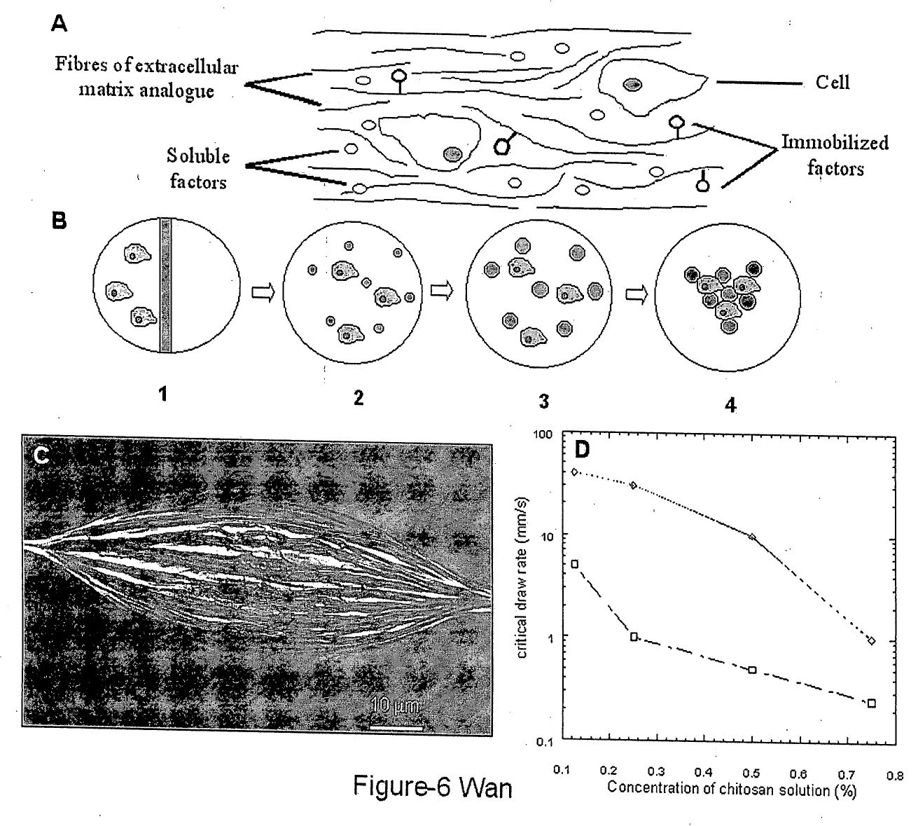

In nature, cells are embedded in an extracellular matrix (ECM) that not only binds them together, but contains the regulatory signals for cell growth, proliferation and death. Chitin, a copolymer of N-acetylglucosamine and glucosamine, and alginate, a copolymer of D-mannuronic and L-guluronic acids are suitable ECM materials for reasons, including; 1. these natural biopolymers mimic the carbohydrate component of the ECM and play a mainly structural role, allowing the selective incorporation of signalling factors; 2. they are polyelectrolytes of opposite charge that can participate in IPC fibre formation. While alginate is naturally water soluble, chitin has to be partially deacetylated to confer water solubility (see, Makromol. Chem. 177, 3589-3600 (1976)) and to generate additional amino groups for ionic complexation. To construct the biostructural unit, (FIG. 6A) the cells, proteins and/or other factors are dispersed into one of the polyelectrolyte phases and drawn up into the fibre. As this occurs, the interface is broken down into many individual “interface microdomains”. (FIG. 6B) These complexed regions are now freed from their kinetic constraint due to mixing with fresh polyelectrolytes, and act as nucleation sites for further complexation. This occurs very rapidly (polyelectrolyte complex formation has been shown to take place in less than 5 μs; see, Doklady Akademii Nauk SSSR 299, 1405-1408 (1988)) to form individual “nuclear” fibres. As these “nuclear” fibres increase in size, the viscosity of the free excess component outside the fibres decreases, due to a decrease in its concentration and an increase in the ionic strength of the medium, which results from the release of counterions. Macromol. Symp. 162, 1-21 (2000). These “nuclear” fibres eventually coalesce around the cells, and the excess polyelectrolytes form gel droplets along the fibre axis. This phenomenon results in a hybrid structure where the cells are closely associated with fibres of the artificial ECM. The coalescence of pre-formed “nuclear” fibers provides a unique mechanism by which the cells can be encapsulated within the fiber without compromising its continuity, enabling the fiber to go “around” them.

The fact that the primary fibre matrix is composed of thinner fibres can be shown experimentally. When nascent fibre was drawn and immediately placed in contact with a glass slide, the gel droplets spread out into two-dimensional bead-like structures. Within these regions, the primary fibre could be seen to fan out into individual fibres of submicron diameter, resulting in an onion-like venation pattern. (FIG. 6C) Support for the fibre formation hypothesis comes from experiments where fibres were drawn at different rates and different concentrations of polyelectrolyte. For each concentration, two critical draw rates could be identified. First, a draw rate existed above which no fibre could be drawn, this was termed the “fibre point”. The “fibre point” corresponds to the initial fibre formation event that gives structural integrity to the fibre form. At draw rates slower than the “fibre point”, a second critical draw rate existed below which a beadless fibre would be formed (“bead point”). The very fact that two critical draw rates were observed indicated that the fibre formation was constituted of two separate phenomena: an initial fibre formation, followed by a second event that resulted in bead formation. Both critical draw rates were inversely proportional to the concentration, and therefore viscosity of the polyelectrolyte solutions. (FIG. 6D) As the hypothesized “nuclear fibre” formation and coalescence are diffusion controlled and also expected to be inversely proportional to solution viscosity, this allows one to relate “nuclear” fibre formation to the “fibre point” and their coalescence to the “bead point”, respectively.

In addition to cells and their associated extracellular matrix, the biostructural unit should also provide for features that allow incorporation of protein molecules, diffusible and/or immobilized, that play signalling or structural roles. Protein can be physically entrapped by dispersing within one of the polyelectrolyte phases, then drawing up into the fibre. In addition to influencing the growth and proliferation of cells within the biostructural unit itself, release of diffusible protein may well influence the development of adjacent tissue structures, as in the case of the paracrine factors involved in embryologic development. See, Cell 84, 127-136 (1996). The value of tissue engineering scaffolds with features of controlled protein release have also been demonstrated. Nat Biotechnol. 19, 1029-1034 (2001). The release kinetics can be adjusted by employing the putative ionic interactions between the fibrous matrix and the protein, charged in accordance with its isoelectric point. Here, it is shown how the release profile of bovine serum albumin is dependent on the concentration ratio of the polyelectrolytes used in fibre formation.

There is strong evidence of benefits in presenting cell-adhesion ligands and growth factors in a tethered manner for tissue engineering. Science 295, 1009 (2002). Conjugation of ligands to preformed scaffolds is difficult and lacks the spatial definition. Although fibrous scaffolds can be derivatized at the fibre level, (see, J. Adhesion Sci. Technol. 16, 1715-1735 (2002)) the physical properties of the fibre may be compromised, especially if the fibre is resorbable. Fibres by interfacial polyelectrolyte complexation, however, can be modified by pre-derivatizing the polyelectrolyte components with ligands prior to the fibre drawing step. These immobilized ligands may be presented to cells within the fibrous matrix, or those attached on the fibre surface.

This method is advantageous in that the ligand density can be controlled by varying the ratio of modified to non-modified polyelectrolyte used for fiber fabrication. Even fragile and highly resorbable fibers can be obtained, whose poor physical properties would preclude “post-fibre” modification. This was illustrated with biotin as the ligand. Immobilization of a wide variety of biotinylated proteins could be achieved by immersing the biotinylated fibres in avidin, followed by protein for a brief period.

The guided alignment and arrangement of cells into tissues by polymeric scaffolds constitute the major, defining principle of tissue engineering. Thus far, the seeding of cells onto preformed scaffolds has been the dominant paradigm in the field. A recent innovation involving the use of confluent cell layers which are built up into 3D-tissue structures without artificial scaffolds demonstrates the advantages of the bottoms-up approach. See, Biomaterials 24(13) 2309-16, (2003). The approach described herein retains the concept of the polymeric scaffold; however, it is based on the biostructural unit which is subsequently assembled into a tissue construct. Such an approach obviates many problems associated with the traditional method of making cell-scaffold constructs. For example, a consistently high and uniform density of encapsulated cells in the final construct could be conveniently achieved, in contrast to typical fibrous scaffolds where the effectiveness of cell-seeding remains an issue. Low cell densities, if deemed desirable as in certain cultures of human embryonic stem cells, would also be a controllable feature.

Constructs can be made from the biostructural units based on textile technology applicable to both dry and wet fibers. See, e.g., Nonwovens & Industrial Textiles 1, 8-10 (2002); Melliand International 6, 229-234 (2000); Chemical Fibres International 5, 587-588 (2000). While fibers that incorporate protein or biological ligands can be air-dried or lyophilized prior to fabric production, fibres that contain encapsulated cells units must be processed under conditions that avoid drying out of the fiber. Such scaffolds have been made by means of hydro-entanglement and ionic crosslinking techniques as described herein.

Constructs assembled from human mesenchymal stem cells could be induced to differentiate along chondrogenic and osteogenic lineages, by application of the appropriate differentiation media. The Live/Dead and WST-1 assays showed that hMSC proliferated and remained viable for more than 8 weeks in all media. Cell clump formation within the fibre was observed within 24 hours of encapsulation. The rapid formation of these small pellets is believed to be advantageous for cell survival and differentiation, in line with the proven benefits of the pellet culture system for mesenchymal stem cells. It has been reported that the interactions between cells, as well as the lack of interaction between cells and substratum, are essential for in vitro chondrogenesis and maintenance of the chondrocyte phenotype. See, J. Cell Biochem 45, 258-260 (1991).

These results confirm the good mass transfer characteristics of the construct, due to minimal cell-surface distance. Prior to this, besides conventional pellet culture, mesenchymal stem cells have been successfully differentiated to chondrocytes only in an alginate layer system, where mass transfer limitations were overcome by the small layer thickness. See, In Vitro Cell Dev Biol Anim. 38, 457-466 (2002). Compared to the latter, the flexibility of the present fibrous system for the construction of three-dimensional structures is clearly an advantage; furthermore, the fibres require no additional membrane support. The ability of the fibre matrix to anchor ECM is also evident by the positive histological staining for several of the matrix components characteristic of cartilage and bone. It can be envisaged how biostructural units of different cell types could be assembled in vitro to form heterotypic scaffolds with spatially defined patterns of cells. Furthermore, the feasibility of growing cells both within and outside the fibres raises the intriguing possibility of using cell encapsulated fibres to mimic the stromal cell layer support for heterotypic co-cultures.

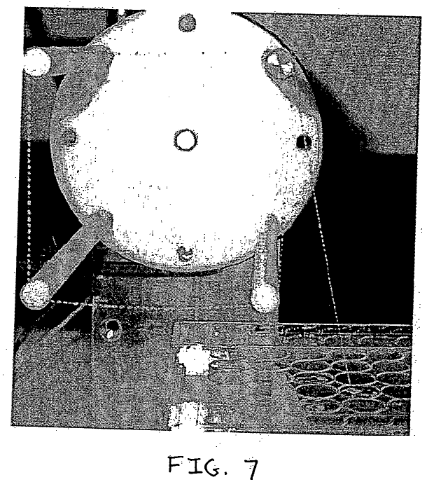

Fiber can be drawn by placing droplets of two oppositely charged polyelectrolyte solutions in close proximity on a level surface, bringing them in contact, then drawing upwards by means of a forceps or bent needle. Fiber can be drawn from the interface until one of the polyelectrolyte phases is depleted, and continuous fiber formation can be achieved by means of a roll-up apparatus (FIG. 7). The process of fiber drawing by interfacial polyelectrolyte complexation is reminiscent of the ‘nylon rope trick’ which illustrates the interfacial polycondensation of polyamides. Journal of Polymer Science Part A-Polymer Chemistry 1996, 34, 531-559. However, a completely different mechanism of fiber formation is involved in interfacial polyelectrolyte complexation, as the present study shows. Unlike interfacial polycondensation, which is essentially a polymerization reaction, interfacial polyelectrolyte complexation is driven by the insolubilization of oppositely charged polyelectrolytes as a result of the neutralization of charges.

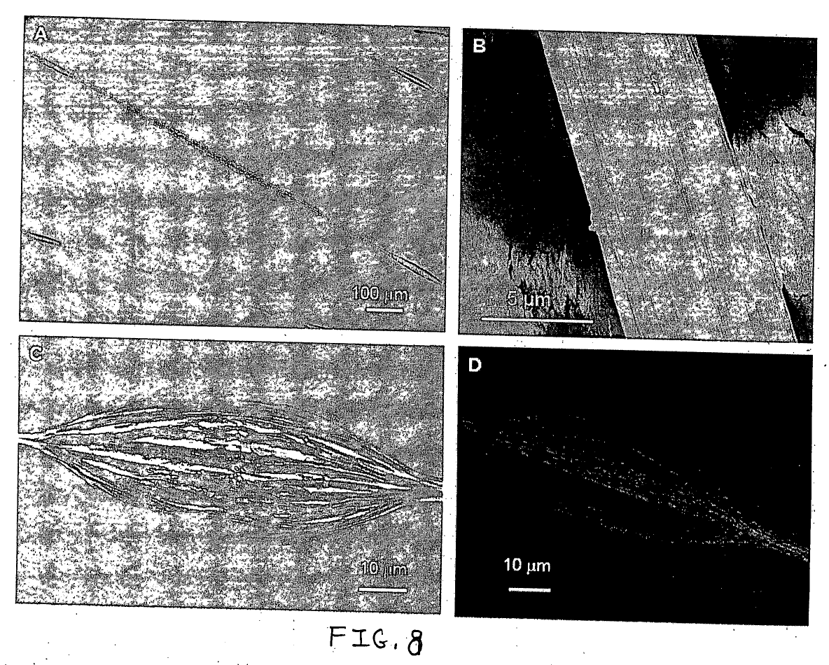

At a glance, the most striking feature of fibers formed via the process of interfacial polyelectrolyte complexation is its unusual morphology of a ‘primary fiber’, with beads spaced out at regular intervals along the fiber axis. These beads take the form of viscous fluid droplets when freshly drawn, which become swellings upon drying (FIG. 8A). The surface of the primary fiber is composed of parallel ridges and valleys, as if it were composed of a conglomerate of finer fibers. (FIG. 8B) This ‘venation’ pattern has also been noted by other workers. J. Appl. Polym. Sci. 2001, 79, 437-446. We could show that the fiber was indeed made up from a collection of thinner fibers. When nascent fiber was drawn and immediately placed in contact with a glass slide, the gel droplets spread out into two-dimensional bead-like structures. Within these regions, the primary fiber could be seen to fan out into individual fibers of submicron diameter, resulting in an onion-like venation pattern (FIG. 9). When quantum dot labeled alginate was used to draw fiber with chitosan, air-dried and visualized using confocal microscopy, regions of fluorescence corresponding to these thinner fibers were observed (FIG. 10D). In the dry form, most of these thinner fibers were observed to traverse the longitudinal axis of the primary fiber. In the bead region, the fluorescence was localized to the fibers and not the surrounding matrix, implying a much higher density of the fiber. (Both fiber and matrix are constituted of a polyelectrolyte complex, deduced by the fact that they are water insoluble even when their original polyelectrolyte solutions are water soluble)

To explore how these finer fibers formed in the IPC process, light microscopy was used to probe the details of the fiber formation process. Firstly, it was clearly seen that a defined interface was always present during fiber drawing, (FIG. 9A) and the nascent fiber emerged from this interface. It could be reasonably deduced that complex was being formed and drawn up in the form of fiber, while fresh polyelectrolyte continuously diffused towards and replenished the interface. Additionally, it was also observed that a fibrillar aggregate or precipitate was continuously being formed, which radiated outward from the fiber-solution junction, and which was not incorporated into the fiber. (FIG. 9B,C) These aggregates eventually disrupted the interface, leading to fiber termination. (FIG. 9D).

A viscous barrier at the junction between the two polyelectrolytes, apparent in the form of a defined interface was deemed important for the formation of fiber by the IPC process. In experiments with different combinations of polyelectrolytes, it appeared that a requisite charge density was essential for fiber formation. Oppositely charged polyectrolytes with lower charge densities did not form a distinct interface region at any concentration, and gradually formed a complex precipitate when mixed. Polyelectrolytes that did form fiber required minimum concentrations in order to form fiber continuously. At lower concentrations, fiber could form, but terminated before all of the polyelectrolyte solution could be depleted, concomitant with the development of a precipitate in the solution.

These observations suggested that the ability to draw fiber continuously was related to a balance between the stability of the interface and the precipitation of a polyionic complex in the solution. To investigate this further, turbidimetric experiments were performed where polyelectrolyte pairs of different concentrations were placed, one above the other in microwell plates. The formation of precipitate, if any, was monitored by the decrease in intensity of light passing through the solutions; precipitation was accompanied by opacity that resulted in a measured absorbance. FIG. 10 illustrates the set of curves that was obtained for combinations of chitosan and alginate concentrations ranging between 0.125 and 0.5% (w/v). Rapid development of turbidity was observed for the lower concentration pairs (Curves A-D). In general, the rate of precipitation was faster at the beginning, and gradually tapered with time. As the concentrations of the polyelectrolytes were increased, the rate of precipitation and quantity of precipitate formed were decreased (Curves E, F). For the highest concentrations used, there was no precipitation at all, within the time frame of the experiment (Curves G-I). For these concentration pairs, the interface was considered to be stable.

The results, in terms of interfacial stability, are summarized in Tables 1 and 2 for the two polyelectrolyte pairs, chitosan-alginate and chitosan-heparin. The ability of the polyelectrolyte pairs to remain in contact indefinitely, without developing any turbidity was a function of both, and not just one of the polyelectrolyte concentrations. This showed that the formation of a barrier towards free mixing of the polyelectrolytes was due to an interaction between the two components, presumably in the form of a complex film at the interface. As compared to alginate, heparin required higher solution concentrations in order to maintain a stable interface with the chitosan solution. This was due to the lower molecular weight of heparin (<30 kD) compared to chitosan (≈300, 000 kD) used in the experiment, thus needing higher concentrations to achieve the requisite viscosity.

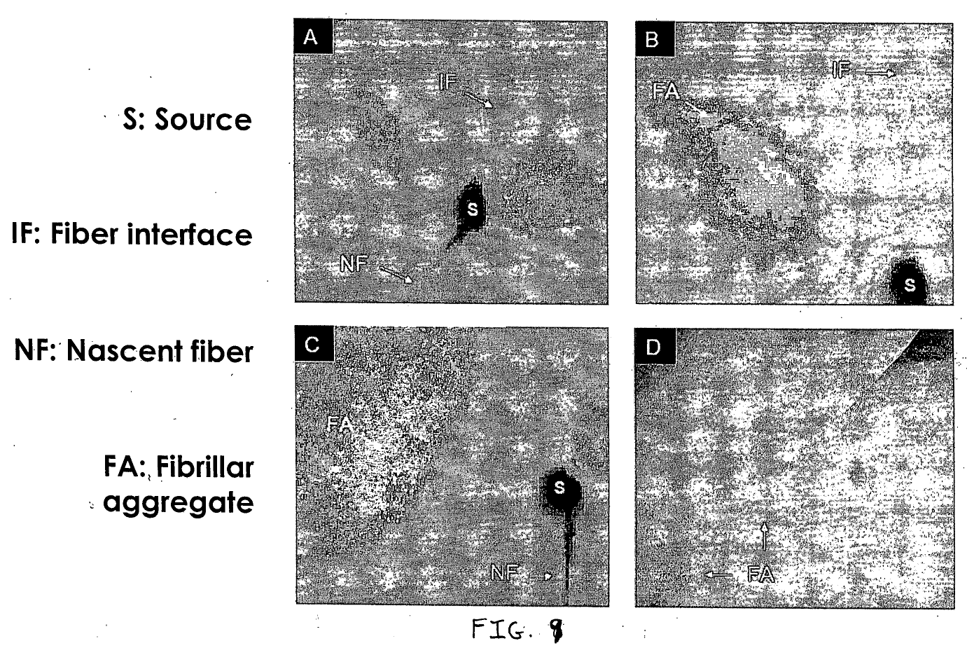

A diagrammatic representation of the interface region, plotted in terms of viscosity versus distance from the interface, is shown in FIG. 11. Ionic complexation of the two polyelectrolytes at, or near the interface results in an increase in the viscosity. This viscous barrier prevents free mixing of the two polyelectrolytes, thus preventing precipitation from occurring. The nascent complex that is removed from this interface by an upward motion can be imagined to form a fiber centered upon the region of maximum viscosity (shaded area). The dimensions of the fibers were demonstrated to be directly related to the area of contact between the two polyelectrolytes, reflecting the interfacial nature of the process. (Table 3) Furthermore, the fiber dimensions were proportional to the concentrations of the polyelectrolytes that were used, a result that is believed to be mediated by solution viscosity (Tables 4,5)

When this viscous polyelectrolyte complex is drawn upwards in a steady nascent stream, simple observation revealed that, within a few seconds, this nascent stream had collapsed into a strand of fiber and its associated droplets. The time that was required for the droplets to form appeared to be inversely proportional to the viscosity of the polyelectrolyte solutions that were used. This bead formation occurred very rapidly, (within 1-2 seconds) and any attempt to measure the formation time directly would have been severely inaccurate, besides relying on subjective visual interpretation. Fortunately, another method was found to quantify the bead formation phenomenon.

Depending on the draw rate, two types of fiber could be formed, “beaded” fiber or “beadless” fiber (Table 6). “Beadless” fiber was formed at low draw rates, due to the coalescence of droplets at the solution-air interface, i.e. the draw rate was slow enough that the droplets had formed before they could leave the solution. The critical draw rate at which beadless fiber formed was defined as the “bead point”. From Table 6, it can be observed that the “bead point” was inversely proportional to the concentration of chitosan solution that was used to draw fiber. In other words, this showed that beads formed more slowly for the more viscous solutions, pointing to a diffusion-dependent mechanism for bead formation.

In addition to the “bead point”, a second critical draw rate could also be identified. This was the draw rate above which no fiber could be drawn, and was termed the ‘fiber point’. The more viscous the solution, the slower the draw rate required in order to draw fiber successfully (Table 6). Thus, the “fiber point” corresponds to the initial fiber formation event that gives structural integrity to the fiber form. As for bead formation, this process occurred more slowly in more viscous solutions, and was also likely to be diffusion-dependent.

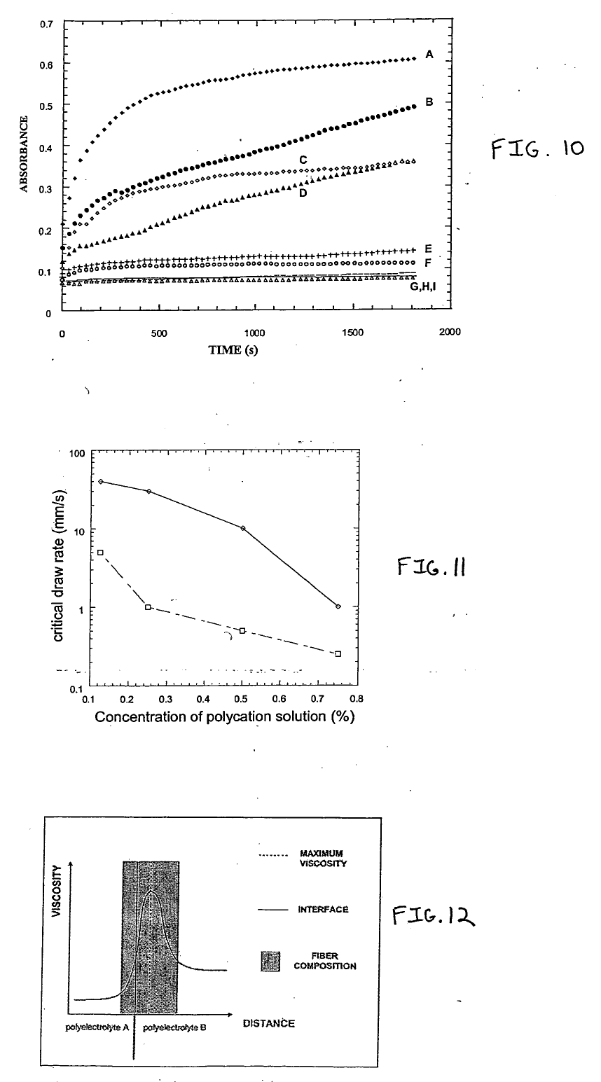

Both critical draw rates were plotted together on the same axes (FIG. 12) The very fact that two critical draw rates were observed indicates that fiber formation is constituted of two separate diffusion-dependent phenomena: an initial fiber formation, followed by a second event that results in bead formation.

Mechanism of Fiber Formation

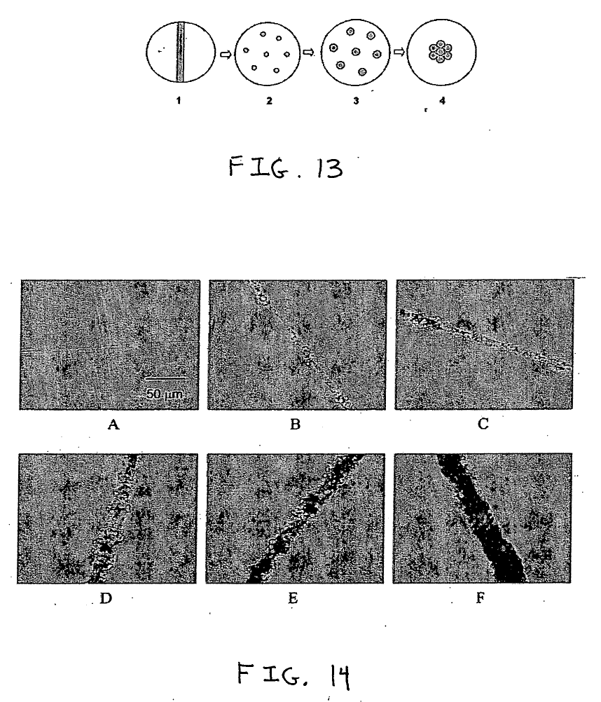

From the experiments and data that have been described, a hypothesis for the mechanism of fiber formation is presented, with reference to FIG. 13. The process can be divided into four stages.

I. Formation of Viscous Barrier at the Interface

The reactants in interfacial polyelectrolyte complexation are both dissolved in an aqueous phase. In order that the complexation remains an interfacial phenomenon, free mixing between the two phases must be avoided. This condition is satisfied by the formation of a polyelectrolyte complex “film” at the interface that acts as a viscous barrier to limit exchange of the polyelectrolytes.

II. Nucleation

As the fiber is drawn, the interface is broken down into many individual, complexed domains. These complexed regions are now freed from their kinetic constraint due to mixing with fresh polyelectrolytes, and act as nucleation sites for further complexation. This occurs very rapidly (polyelectrolyte complex formation has been shown to take place in less than 5 μs, Doklady Akademii Nauk 1988, 299, 1405-1408) to form individual “nuclear” fibres.

III. Growth of “Nuclear Fibres”

As these “nuclear” fibers increase in size, the viscosity of the free excess component outside the fibers decreases, due to a decrease in its concentration and an increase in the ionic strength of the medium, which results from the release of counterions. Macromol. Symp. 2000, 162, 1-21.

IV. Coalescence of “Nuclear Fibers”

These “nuclear” fibers eventually coalesce, and the excess polyelectrolytes form gel droplets along the fiber axis.

As “nuclear fiber” formation and growth and “nuclear fiber” coalescence are diffusion controlled and expected to be inversely proportional to solution viscosity, this allows us to relate “nuclear” fiber formation/growth to the “fiber point” and their coalescence to the “bead point” respectively.

Encapsulation of Materials

The IPC fiber process is unique in its ability to encapsulate materials at ambient temperature and under aqueous conditions, a feature which is especially useful for the encapsulation of biologics. The mode of IPC fibre formation which is hypothesized to involve thin ‘nuclear’ fibers that coalesce provides a mechanism by which particulate materials can be encapsulated within the fiber without unduly compromising its physical properties, enabling the fiber to go ‘around’ the particles. In other fiber types, any trapped particles would effectively reduce the fiber cross section and reduce its mechanical strength, if not terminating it completely.

For the encapsulation experiments, a water-soluble chitosan of degree of deacetylation approximately 50% was used, as it could be dissolved in pure water. In contrast, dissolution of chitosan with higher degrees of deacetylation requires low pHs, which may be deleterious to some of types of encapsulants, for example, cells. The encapsulation feature of the fiber process was illustrated using silica gel. Encapsulation was performed by dispersing silica gel in alginate solution, then drawing fiber against a purely aqueous chitosan solution. FIG. 14 shows the appearance of silica gel encapsulated fiber obtained using different concentrations of the silica gel suspension in alginate. As the silica gel concentration (w/v alginate solution) was increased, a thicker fiber diameter resulted, with an accompanying increase in the quantity of dispersed phase per unit length of fiber. A greater than 10-fold increase in fiber diameter resulted when silica gel was encapsulated at a particle density of 150 mg/mL of alginate solution. Dispersion of silica gel in alginate solution increases its viscosity in proportion to the concentration, and broadens the viscous region of the interface depicted in FIG. 11. This produces an effect similar to increasing the concentration of alginate, leading to a thicker fiber.

The use of biostructural units introduces a new paradigm in tissue engineering. Constructs assembled from these units allow the creation of a highly defined and patterned structure with respect to different cell types and factors. These could be used to investigate normal, or abnormal (cancer, genetic disease) tissue development, and to further our understanding of tissue regeneration.

II. Compounds

Another embodiment is a method of making a fiber composition including a compound herein.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., formulation into therapeutic products, intermediates for use in production of therapeutic compounds, isolatable or storable intermediate compounds, treating diseases, disorders, or symptoms thereof, including those delineated herein). The compounds produced by the methods herein can be incorporated into compositions, including solutions, capsules, cremes, or ointments for administration to a subject (e.g., human, animal). Such compositions (e.g., pharmaceuticals) are useful for providing to the subject desirable health or other physiological benefits that are associated with such compounds.

The compounds of the formulae herein are available from commercial sources or may be synthesized using reagents and techniques known in the art, including those delineated herein. The chemicals used in the synthetic routes may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The chemicals used in the aforementioned methods may include, for example, solvents, reagents, catalysts, protecting group and deprotecting group reagents and the like. The methods described above may also additionally include steps, either before or after the steps described specifically herein, to add or remove suitable protecting groups in order to ultimately allow synthesis of the compound of the formulae described herein. The methods delineated herein contemplate converting compounds of one formula to compounds of another formula. The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, crystallization, chromatography). Other embodiments relate to the intermediate compounds delineated herein, and their use in the methods (e.g., treatment, synthesis) delineated herein.

III. Methods of Treatment

In one embodiment, the present invention provides methods of treating disease and/or disorders or symptoms thereof which comprise administering a composition herein to a subject (e.g., a mammal such as a human).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The preferred therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof.

For therapeutic applications, the compounds of the formulae herein may be suitably administered to a subject such as a mammal, particularly a human, alone or as part of a pharmaceutical composition, comprising the formulae herein together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for administration via fiber scaffolds herein, including rectal, topical (including buccal and sublingual), vaginal or parenteral (including subcutaneous, intramuscular, intravenous and intradermal) administration. Other formulations may conveniently be presented in unit dosage form, or in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

A “pharmaceutically acceptable derivative or prodrug” means any pharmaceutically acceptable salt, ester, salt of an ester, or other derivative of a compound of this invention which, upon administration to a recipient, is capable of providing (directly or indirectly) an active compound of this invention. Particularly favored derivatives and prodrugs are those that increase the bioavailability of the compounds of this invention when such compounds are administered to a mammal (e.g., by allowing an administered compound to be more readily absorbed into the blood) or which enhance delivery of the parent compound to a biological compartment (e.g., the brain or central nervous system) relative to the parent species. Preferred prodrugs include derivatives where a group which enhances solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214.

The compounds of this invention may be modified by appending appropriate functionalities to enhance selective biological properties. Such modifications are known in the art and include those which increase biological penetration into a given biological compartment (e.g., central nervous system), increase bioavailability, increase solubility to allow administration by injection, alter metabolism and alter rate of excretion. It will be appreciated that actual preferred amounts of a given compound herein used in a given therapy will vary according to the particular active compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests, or by any method known in the art or disclosed herein.

The compounds herein may contain one or more asymmetric centers and thus occur as racemates and racemic mixtures, single enantiomers, individual diastereomers and diastereomeric mixtures. All such isomeric forms of these compounds are expressly included in the present invention. The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds herein are expressly included in the present invention. All crystal forms and polymorphs of the compounds described herein are expressly included in the present invention.

Therefore, in certain embodiments, compounds of the invention, such as those of the formulae herein, are administered at dosage levels of about 0.0001 to 4.0 grams once per day (or multiple doses per day in divided doses) for adults. Thus, in certain embodiments of this invention, a compound herein is administered at a dosage of any dosage range in which the low end of the range is any amount between 0.1 mg/day and 400 mg/day and the upper end of the range is any amount between 1 mg/day and 4000 mg/day (e.g., 5 mg/day and 100 mg/day, 150 mg/day and 500 mg/day). In other embodiments, a compound herein, is administered at a dosage of any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 2 mg/kg/day, 5 mg/kg/day and 20 mg/kg/day). The dosing interval can be adjusted according to the needs of individual patients. For longer intervals of administration, extended release or depot formulations can be used.

IV. Kits

The invention also provides kits for making a fiber composition herein, including a fiber comprising at least two polyionic fiberelles and a bioactive material. In one embodiment, the kit includes an effective amount of a compound herein in unit dosage form, together with instructions for administering the compound to a subject suffering from or susceptible to a disease or disorder or symptoms thereof. In other embodiments, the kit comprises a sterile container which contains the reagents suitable for making the fiber compositions herein; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments. The instructions will generally include information about the use of the reagents of the formulae herein for making a fiber composition herein, including those of having bioactive agents. In other embodiments, the instructions include at least one of the following: description of the reagent(s); stoichiometry or drawing schedule; bioactive agents or compositions thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The recitation of an embodiment for a variable herein includes that embodiment as any single embodiment or in combination with any other embodiments or portions thereof. Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein

The invention will be further described in the following examples. It should be understood that these examples are for illustrative purposes only and are not to be construed as limiting this invention in any manner.

### EXAMPLES

One representative example of the fiber for protein and cell encapsulation employed water soluble chitin (WSC) prepared using chitin from crab shell (Aldrich) as the starting material, and sodium alginate (low molecular weight, Sigma). The degree of deacetylation of chitin was measured to be 56% by potentiometric titration. A 2% sodium alginate solution possessed a viscosity of approximately 250 cps at 25° C. For the determination of critical draw rates, chitosan (low molecular weight, degree of deacetylation: 75-85%, Aldrich) was employed. A 1% chitosan solution in 1% acetic acid possessed a viscosity of 20-200 cps.

### Example 1

Fiber Fabrication.

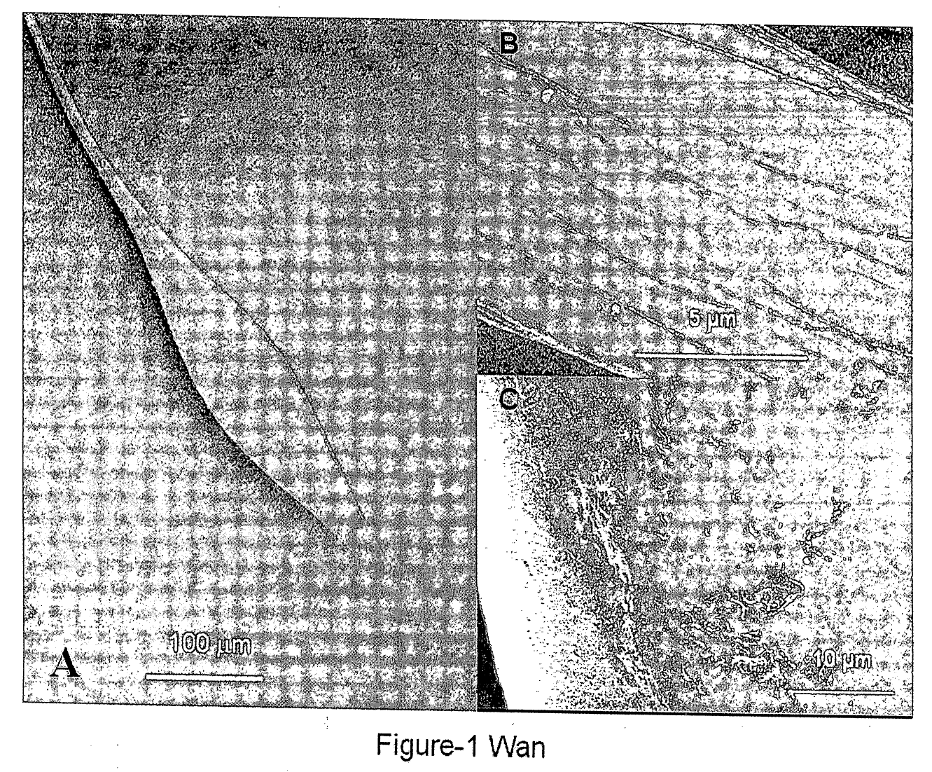

Fiber was fabricated in the following way: two polyelectrolyte solutions were placed in close proximity on a level surface, then brought into contact by means of forceps or a needle. The mixture in the region of the interface was then scooped up in a continuous upward motion to create a fiber, which could be drawn continuously until one of the polyelectrolyte solutions was depleted. A scanning electron micrograph of a typical fiber is shown in FIG. 1. The fiber surface exhibits a pattern of parallel ridges, as if it were composed of a conglomerate of finer fibers. Interestingly, beads are present at regular intervals along its axis. In contrast to its polyelectrolyte precursors, the fiber is water insoluble.

Fiber was fabricated by drawing up the interface between two oppositely charged polyelectrolyte solutions using a bent syringe needle (25G3/8) attached to the slider of a linear motor (LinMot, Switzerland) with a stroke length of 30 cm. A LinMot Talk software allowed drawing to be performed according to pre-programmed motion profiles. The solution interface was created by first placing droplets of the polyelectrolyte solutions 1-2 mm apart on the surface of a polystyrene or Teflon plate. Droplet volumes were commonly in the range of 1-10 μL. The bent needle was used to bring the two droplets in contact and the upward drawing motion was instantly commenced. For encapsulation of protein and silica gel, draw rates of 10 mm/s and 20 mm/s were employed, respectively.

For the measurement of critical draw rates, another configuration was employed where a droplet of alginate solution was placed above a droplet of chitosan in 0.15 M acetic acid at a volume ratio of 1 to 2.5, respectively. A syringe needle attached to the LinMot slider was inserted to touch the plate surface and gently aspirated to consume a small volume of the lower polyelectrolyte. The slider motion was then commenced at different draw rates. The alginate concentration was kept at 1% while the chitosan concentration was varied.

### Example 2

Encapsulation and Release of Protein.

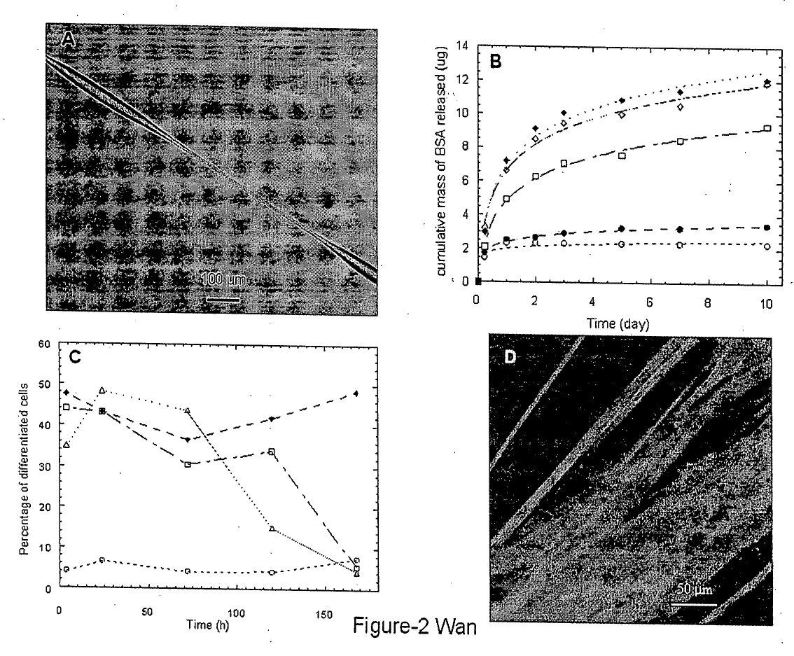

Bovine serum albumin (BSA) was encapsulated in water soluble chitin-alginate (WSC-A) fibers at different polyelectrolyte concentration ratios. The protein was uniformly distributed, as evident from the light microscope image of encapsulated FITC-BSA. (FIG. 2A) Even after washing, a protein loading level of at least 40% (mass protein/mass polymer) could be achieved. This loading level is considerably higher than that typically obtained by conventional solvent evaporation techniques for formulation of controlled release microspheres. Since the solution goes completely into fiber formation, the efficiency of protein encapsulation is close to 100%.

The cumulative release profiles of BSA from the fiber are shown in FIG. 2B, typical of a diffusion-controlled release mechanism. As the chitin to alginate concentration ratio increased, a more sustained release of protein was observed. This can be explained by the net negative charge of BSA at neutral pH. It follows that a fiber with more positive charges (higher chitin/alginate ratios) would be able to bind BSA more effectively and release it over a longer period of time. For the latter case, protein release was measurable for a period of at least 2 weeks.

For a WSC-alginate concentration ratio of 1:2, fibers were drawn using 5 μL of a 1% alginate solution containing 25 mg/mL BSA, and 12.5 μL of 0.5% WSC solution. Five nascent fibers were spooled per Teflon ring (Fisher Scientific) and air-dried overnight. The fibers were washed with phosphate buffered saline (PBS) for 3 hours to remove adsorbed protein and protein encapsulated in the low density bead regions of the fibers. To measure the protein release profile, spooled fibers were placed in wells of a 24-well tissue culture plate containing pH 7.4 PBS, which were then placed in an incubator at 37° C. At fixed time intervals, supernatants were sampled and replaced with the same volume of fresh buffer. For time periods of up to 2 days, 1 mL of incubation media was used, this was subsequently reduced to 600 uL.

Bioactivity of Protein.

Nerve growth factor (NGF) was used as the model protein to investigate if incorporation of protein into the fibre compromises its bioactivity. The bioactivity of the released NGF was confirmed by its ability to differentiate PC12 cells, and in a dose-dependent manner. (FIG. 2C) At the increasing loading concentrations of NGF indicated, significant cell differentiation could be observed for time periods of up to 3 d, 5 d and 7 d, respectively. In all cases, the minimum concentration of NGF required to induce neurite outgrowth appeared to be in the region of 0.5 ng/mL, which agrees well with the literature. See, Physiological Reviews 60, 1284-1335 (1980).

Encapsulation of recombinant human β-NGF (MW 13,200, R&D Systems) was performed in the same way as described for BSA. Ring-spooled fibers were incubated in RPMI media over a period of 1 week, during which supernatants were collected for the PC12 bioassay and NGF ELISA (DuoSet ELISA Development System for human β-NGF, R&D Systems).

### Example 3

Encapsulation of Cells.

Cells encapsulated in the fiber retained good viability. Two primary cell lines of human dermal fibroblasts (HDF) and bovine pulmonary artery endothelial cells (BPAEC) were encapsulated in WSC-A fibers. Cell viability was established by means of a Live/Dead viability/cytotoxicity kit (Molecular Probes) and a WST-1 assay (Roche Diagnostics GmbH). The WST-1 assay for BPAEC gave relative absorbances of 100%, 95%, 96% and 98% at days 2, 5, 7 and 10 respectively, reflecting the activity of viable cells. The relative absorbances for HDF were 100%, 91%, 74% and 82% for the same time periods.

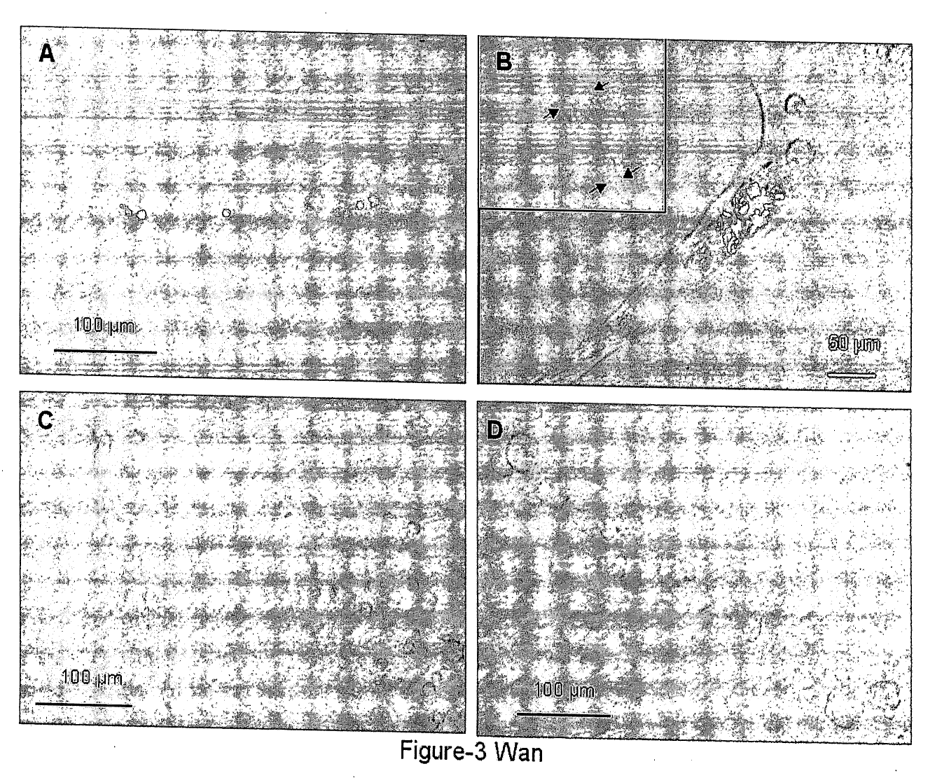

In contrast to their morphology in monolayer culture, encapsulated HDF and BPAEC were more spherical in shape (FIG. 3A,B). Cells could be entrapped within both bead and fiber regions, and remain viable. Cell clumps in the fiber region appeared as if they were prying open thinner fibers that composed the main fiber, enlarging its diameter significantly.

A third cell type, the human mesenchymal stem cells (hMSCs) were also studied. The hMSCs encapsulated in the fibre at high density characteristically formed clumps within 24 hours. (FIG. 3C,D) Similar to HDF and BPAEC, the phenomenon of thinner fibers within the main fiber that accommodated the size and geometry of the cell aggregates could be observed.

Human dermal fibroblast (HDF) and bovine pulmonary artery endothelial cell (BPAEC) pellets containing 2×10⁵ and 8×10⁵ cells, respectively, were first dispersed in 20 uL of 1% alginate by tituration, followed by a brief period of vortexing. Five uL of the cell-alginate suspension was transferred onto a sterile polystyrene surface and 10 uL of 0.5% water-soluble chitin placed in close proximity but not contacting it. A pair of forceps was then used to bring the polyelectrolyte droplets in contact while simultaneously drawing up the solution in a smooth motion. The drawn fiber was spooled around a Teflon ring and placed immediately into the well of a 24-well plate containing DMEM medium (10% FBS, 1% GPS). For confocal microscopy, ring-spooled fibers were placed in a coverslip chamber (Nunc).

### Example 4

Immobilization of Protein.

Biotinylation of fibre allows convenient attachment of desired proteins to the fibre via an avidin bridge. Alginate was first biotinylated, and then used to form fibre with water-soluble chitin. The biotinylated fibre was treated with 0.3 mg/mL avidin solution for 15 minutes and rinsed with phosphate buffered saline to remove excess avidin. Treatment of this fiber with biotinylated NGF followed by immuno-fluorescence labeling yielded a fiber that was intensely fluorescent as seen by confocal microscopy. (FIG. 2D) This indicated a high density of biotin on the surface of the fiber, access of this biotin to avidin, and the availability of additional binding sites on avidin for the binding of the biotinylated NGF.

Biotinylation of Sodium Alginate

2.5 mg of sodium alginate (low molecular weight, Sigma) was dissolved in 0.5 mL of 0.1 M MES buffer, pH 5.5. 104.8 μL of a solution of 36 mg/mL Biotin-XX-hydrazide (Calbiochem) in dimethylsulfoxide was added to the alginate solution and vortexed briefly to mix. 12.5 μL of a 0.12 mg/μL solution of freshly prepared 1-ethyl-3-(3-dimethylaminopropyl)carbodiimide (Sigma) solution in 0.1 M MES buffer, pH 5.5 was added to the first solution containing alginate and Biotin-XX-hydrazide. The reaction mixture was incubated for at least two hours at room temperature. The product was purified using a dialysis membrane with a molecular weight cutoff of 3500 kD (Pierce). The molar ratio of biotin to alginate was determined using the HABA assay (Pierce), yielding a value of one biotin to every 19 alginate repeating units.

Biotinylation of NGF:

One hundred μL of 100 μg/mL NGF, reconstituted in 0.1% BSA in PBS, and 0.35 mg of biotin-PEG-NHS (biotin-terminated poly(ethylene glycol)-N-hydroxysuccinimide, MW 3400, Shearwater Polymers, 30-fold in molar excess) were added to 1 mL of PBS. The conjugation reaction was carried out for 24 hours at 4° C. To remove unreacted biotin-PEG-NHS molecules and unbiotinylated NGF, the reaction solution was added into a Microcon Centrifugal filter device (3000 NMWL) and centrifuged 3 times with 1 mL of PBS. The molar ratio of biotin to NGF was determined by the HABA assay to be 15.9.

Immuno-Fluorescence Detection of Immobilized NGF:

The NGF-immobilized fiber was washed with PBS before blocking with 2% BSA in PBS for 20 minutes. The fiber was washed twice with 1% BSA solution and incubated with 400 μL of 2.5 μg/mL anti-human β-NGF goat IgG antibody (R & D Systems) overnight at 4° C. After washing, the samples were incubated with TRITC-conjugate anti-goat IgG antibody (Sigma) for 2 hours. The samples were washed twice with 1% BSA before confocal imaging.

### Example 5

Assembly of Biostructural Units into Constructs.

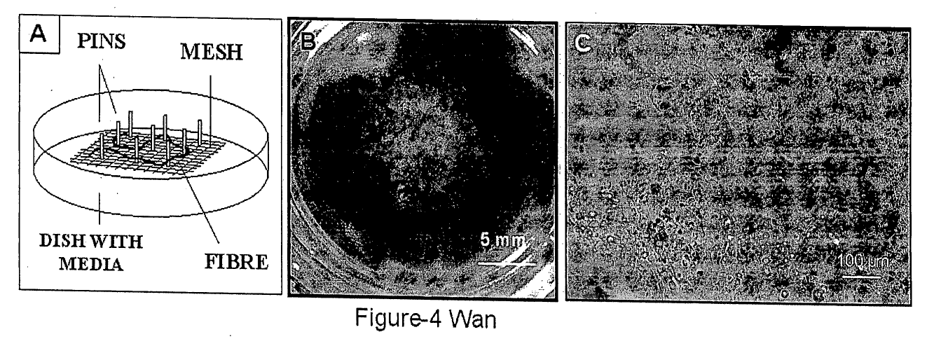

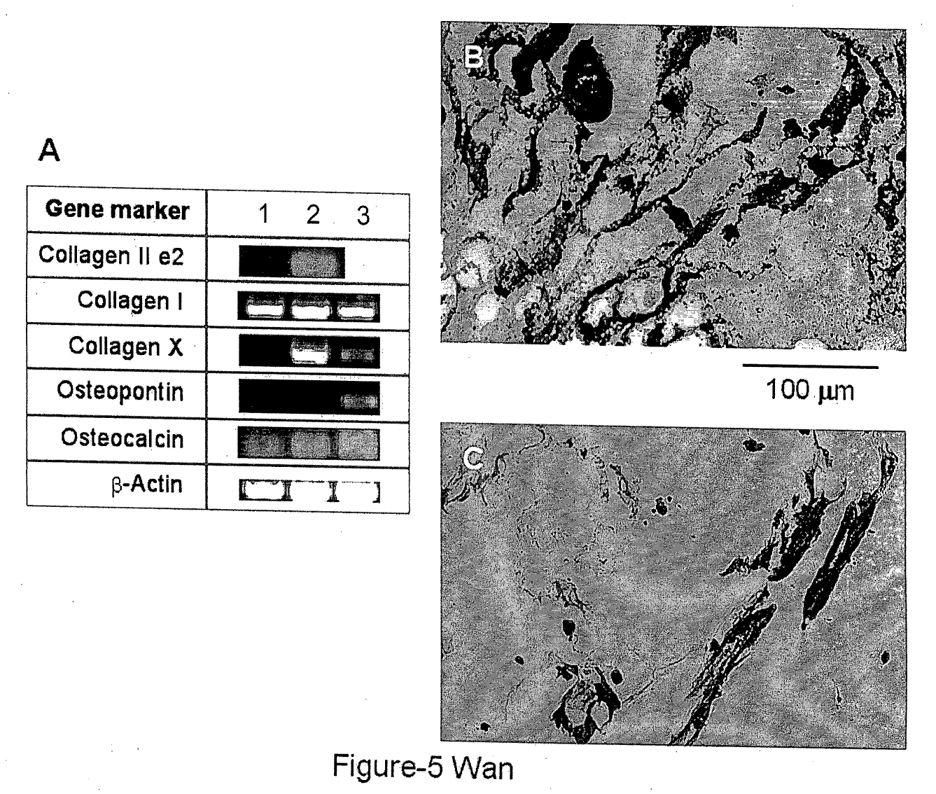

FIG. 4 illustrates how the biostructural units could be assembled to form a construct. Constructs were grown in hMSC media for a period of 3 weeks, after which they were switched to chondrogenic and osteogenic media respectively, for an additional three weeks. At the end of the culture period, RT-PCR showed that cells in the chondrogenic and osteogenic media expressed mRNA markers of the chondrogenic (collagen I, II e2 and X) and osteogenic (collagen I and X, osteopontin and osteocalcin) phenotypes respectively (FIG. 5A) Positive staining of von Kossa and alizarin red S were observed for the osteogenic sample, indicating calcium deposition. (FIG. 5B,C) Additionally, the latter exhibited positive alkaline phosphatase activity, as compared to controls. All samples were stained positive with both alcian blue and toluidine blue, indicating the presence of proteoglycans.

Scaffold Formation from Wet Fibres by Hydroentanglement:

Fibers were drawn, immersed in 5 mM calcium chloride for five seconds, and washed in PBS. The ionically crosslinked fiber was laid down on a 5 cm×5 cm fiberglass screen which was immediately placed into deionised water. This procedure was repeated until an approximately 5 mm thick web had formed. This newly formed web was folded onto itself (on the fiberglass screen) and subjected to water jets at a pressure of 8,900 kPa by means of a pressure washer (Karcher 240). The gun of the washer was maintained at 10 cm above the sample and moved at a fixed speed along the sample as water jets were applied. To achieve a stable uniform structure, water jets were applied to both sides of the sample.

Scaffold Formation from Wet Fibres by Ionic Crosslinking:

Human mesenchymal stem cells (hMSC) were encapsulated by dispersing a cell pellet containing 1.6×10⁶ cells in 80 μL 0.3% sodium alginate (PRONOVA™ MVM, FMC BioPolymer) by tituration, then drawing fiber against 0.5% WSC. Nascent fiber was immediately immersed in 5 mM calcium chloride for 15 seconds, then randomly wound around stainless steel pins arranged in a “3×3” matrix above a removable mesh, in MSCGM medium (5% FBS, 2% L-glutamine, 0.1% penicillin/streptomycin, Cambrex). For every three crosslinked fibers laid down in this manner, one non-crosslinked fiber was interspersed. After sixteen fibers had been laid down, the construct was removed from the media with the aid of the mesh, and transferred to fresh media contained in a 6-well plate with 24 mm diameter transwell inserts (Costar, Corning). Scaffolds constructed via the hydro-entanglement and ionic crosslinking processes, and the apparatus for laying down the wet fibers in the latter process are described. The appearance and distribution of cells and ECM in the fiber were analyzed by confocal microscopy and routine histological methods.

### Example 6

Effect of Interfacial Area and Polyelectrolyte Concentration on Fiber Dimensions

The interfacial area of contact between the two polyelectrolyte solutions was defined by using Teflon channels of varying cross-sectional areas while maintaining the chitosan concentration at 1% (w/v) and alginate concentration at 0.5% (w/v). The influence of alginate and chitosan solutions on fiber dimensions was investigated by drawing fiber from an interfacial area of 3 mm².

Effect of Viscosity on the Stability of the Interface

100 μL of various concentrations of chitosan solution were added to a 96-well plate, and 50 μL of alginate or heparin solution was carefully introduced into each of the heparin-containing wells, via the side of the well. The 96-well plate was placed into a microplate reader (Bio-Rad, Model 550) and measurement of the absorbance at 450 nm was immediately commenced, at 30 second sampling intervals. The turbidity profile for each sample was obtained in terms of absorbance vs. time. The concentrations of chitosan and alginate/heparin solutions used are shown in Tables 1 and 2. Three samples were measured for each concentration pair to obtain statistical significance.

Light Microscopy of Fiber Drawing

Ten μL of 0.125% chitosan solution was placed approximately 2 mm from 10 μL of 1% alginate solution on a glass slide staged on an optical microscope. Using a pair of forceps, the solutions were brought together and fiber was produced by pulling the forceps upwards, away from the solution. A camera was mounted onto the light microscope to capture the images of fiber being drawn from the solution interface, at a magnification of 100×.

Silica Gel Encapsulation

Chitosan with a degree of deacetylation of 54% was prepared by the partial deacetylation of chitin (practical grade, Sigma), see Makromol. Chem. 1976, 177, 3589-3600. Silica gel (Aldrich, mean particle diameter of 6 μm, 70-230 mesh) was dispersed in alginate solution and fiber was drawn at a draw rate of 20 mm/s using the linear motor (LinMot).

TABLE 1

Heparin concentration (% w/v)

| | | 0.5 | 1.0 | 1.5 | 2.0 |
|---|---|---|---|---|---|
| Chitosan | 0.125 | ● | ● | ● | ● |
| concentration | 0.25 | ● | ● | ● | ● |
| (% w/v) | 0.3 | ● | ● | ◯ | ◯ |
| | 0.4 | ● | ◯ | ◯ | ◯ |
| | 0.5 | ◯ | ◯ | ◯ | ◯ |
| | 0.6 | ◯ | ◯ | ◯ | ◯ |

The stability of the interface between chitosan and alginate solutions of various concentrations, as indicated by the development or absence of turbidity; ●: turbid (unstable); ◯: clear (stable).

TABLE 2

Alginate concentration (% w/v)

| | | 0.125 | 0.25 | 0.5 | 1.0 |
|---|---|---|---|---|---|
| Chitosan | 0.125 | ● | ● | ● | ◯ |
| concentration | 0.25 | ● | ● | ◯ | ◯ |
| (% w/v) | 0.3 | ● | ◯ | ◯ | ◯ |
| | 0.4 | ● | ◯ | ◯ | ◯ |
| | 0.5 | ● | ◯ | ◯ | ◯ |
| | 0.6 | ● | ◯ | ◯ | ◯ |

The stability of the interface between chitosan and heparin solutions of various concentrations, as indicated by the development or absence of turbidity; ●: turbid (unstable); ◯: clear (stable).

TABLE 3

| | Area of interface(mm²) | | | | |
|---|---|---|---|---|---|
| | 0.5 | 1 | 3 | 4 | 5 |
| Bead diameter | 1.14 ± 0.34* | 1.84 ± 1.16 | 2.20 ± 0.10 | 2.50 ± 0.25 | 2.32 ± 0.33 |
| (μm) | | | | | |
| Fiber diameter | 7.00 ± 0.54 | 11.93 ± 1.16 | 24.80 ± 0.89 | 30.50 ± 2.73 | 29.52 ± 4.21 |
| (μm) | | | | | |

The dependence of fiber dimensions on the interfacial area between two polyelectrolyte solutions.

TABLE 4

| | Alginate concentration (% w/v) | | | | |
|---|---|---|---|---|---|
| | 0.5 | 0.75 | 1 | 1.25 | 1.5 |
| Bead diameter | 1.28 ± 0.35* | 1.86 ± 0.44 | 2.20 ± 0.10 | 2.19 ± 0.25 | 2.20 ± 0.32 |
| (μm) | | | | | |
| Fiber diameter | 6.59 ± 4.47 | 14.49 ± 1.33 | 24.80 ± 0.89 | 21.30 ± 2.24 | 21.40 ± 2.89 |
| (μm) | | | | | |

The dependence of fiber dimensions on the alginate solution concentration.

TABLE 5

| | Chitosan concentration (% w/v) | | | |
|---|---|---|---|---|
| | 0.125 | 0.25 | 0.5 | 0.75 |
| Bead | 2.13 ± 0.07* | 2.30 ± 0.29 | 2.20 ± 0.10 | 2.06 ± 0.20 |
| diameter | | | | |
| (μm) | | | | |
| Fiber | 10.75 ± 0.66 | 15.72 ± 1.27 | 24.80 ± 0.89 | 27.19 ± 0.45 |
| diameter | | | | |
| (μm) | | | | |

The dependence of fiber dimensions on the chitosan solution concentration.

TABLE 6

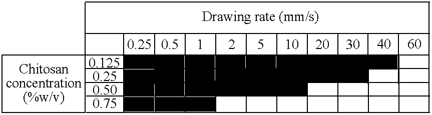

Ability to draw fiber, and type of fiber formed at various draw rates and concentrations of chitosan solution. Shaded region indicates the draw rates at which fiber could be drawn successfully; in the darker region, a beadless fiber was drawn.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

A number of embodiments of the invention have been described. Nevertheless, it will be understood that various modifications may be made without departing from the spirit and scope of the invention. Accordingly, other embodiments are within the scope of the following claims.

What is claimed:

1. A fiber comprising at least two polyionic fiberelles and at least one bioactive material encapsulated within the fiber.

2. The fiber of claim 1, wherein the polyanionic fiberelles are composed of at least one polycationic polymer and at least one polyanionic polymer.

3. The fiber of claim 2, wherein the polycationic polymer is biodegradable or biocompatible.

4. The fiber of claim 3, wherein the polycationic polymer is selected from the group consisting of natural and synthetic carbohydrate or polypeptide polymers having a net positive charge.

5. The fiber of claim 2, wherein the polyanionic polymer is biodegradable or biocompatible.

6. The fiber of claim 5, wherein the polyanionic polymer is selected from the group consisting natural and synthetic carbohydrate or polypeptide polymers having a net negative charge.

7. The fiber of claim 2, wherein the polycationic polymer is chitin or chitosan, and the polyanionic polymer is selected from the group consisting of alginate, gellan, or a combination thereof.

8. The fiber of claim 2, wherein the polycationic polymer is chitin and the polyanionic polymer is alginate.

9. The fiber of claim 1, wherein the bioactive material is selected from drugs, proteins, DNA, RNA, cells, viruses, microparticles, nanoparticles, contrast agents, or combinations thereof.

10. The fiber of claim 9, wherein the bioactive material provides at least one extracellular matrix molecule suitable for stabilizing cells.

11. The fiber of claim 10, wherein the extracellular matrix molecule is a protein or drug capable of recapitulating an extracellular matrix of natural tissue.

12. The fiber of claim 1, wherein the fiber comprises between two and 10,000 fibrelles.

13. The fiber of claim 1, wherein the fiber comprises between 10 and 1000 fibrelles.

14. The fiber of claim 1, wherein the fiber has a substantially homogenous cross-section.

15. The fiber of claim 1, wherein the cross-section is between about 0.5 and about 10 μm.

16. The fiber of claim 1, wherein the fiber comprises domains of narrow cross-section interposed with beads having a larger cross-section.

17. The fiber of claim 16, wherein the bead has a cross-section between about 2 and about 25 times the cross-section of the narrower domains.

18. An article of manufacture comprising at least one fiber of any one of claims 1-17.

19. The article of manufacture of claim 18, wherein the article is selected from braids, woven and non-woven fabrics, mesh, and combinations thereof.

20. A tissue engineering scaffold comprising at least one fiber comprising at least two polyionic fiberelles and at least one bioactive material encapsulated within the fiber.

21. The tissue engineering scaffold of claim 20, wherein the bioactive material is selected from drugs, proteins, DNA, RNA, cells, viruses, microparticles, nanoparticles, contrast agents, or combinations thereof.

22. The tissue engineering scaffold of claim 21, wherein the bioactive material provides an extracellular matrix suitable for stabilizing cells.

23. The tissue engineering scaffold of claim 22, wherein the bioactive material is a protein or drug associated with tissue regeneration.

24. A method of preparing a fiber comprising at least two polyionic fiberelles and at least one bioactive material encapsulated within the fiber, the method comprising the steps of:
(a) providing an aqueous solution of a polyanionic polymer and an aqueous solution of a polycationic polymer, wherein at least one of the polyanionic polymer solution or the polycationic polymer solution further comprises at least one bioactive material;
(b) contacting the polyanionic polymer solution and the polycationic polymer solution under conditions conducive to form an interface; and
(c) pulling a fiber from the interface.

25. The method of claim 24, wherein the interface between the polyanionic polymer solution and the polycationic polymer solution has a cross-section of less than about 10 mm².

26. The method of claim 25, wherein the cross-section of the interface is between about 1 mm² and about 5 mm².

27. The method of claim 24, wherein the fiberelles are composed of at least one polycationic polymer and at least one polyanionic polymer.

28. The method of claim 27, wherein the polycationic polymer is biodegradable or biocompatible.

29. The method of claim 24, wherein the polyanionic polymer solution has a polyanionic polymer concentration of less than about 10% (w/v).

30. The method of claim 29, wherein the polycationic polymer is selected from the group consisting of natural and synthetic carbohydrate or polypeptide polymers having a net positive charge.

31. The method of claim 24, wherein the polyanionic polymer is biodegradable or biocompatible.

32. The method of claim 31, wherein the polyanionic polymer is selected from the group consisting natural and synthetic carbohydrate or polypeptide polymers having a net negative charge.

33. The method of claim 24, wherein the polycationic polymer is chitin or chitosan, and the polyanionic polymer is selected from the group consisting of alginate, gellan, or a combination thereof.

34. The method of claim 24, wherein the polycationic polymer is chitin and the polyanionic polymer is alginate.

35. The method of claim 24, wherein the bioactive material is selected from drugs, proteins, DNA, RNA, cells, viruses, microparticles, nanoparticles, contrast agents, or combinations thereof.

36. The method of claim 35, wherein the bioactive material provides at least one extracellular matrix molecule suitable for stabilizing cells.

37. The method of claim 36, wherein the extracellular matrix molecule is a protein or drug capable of recapitulating an extracellular matrix of natural tissue.

38. The method of claim 24, wherein the pulling of the fiber from the interface comprises a substantially continuous linear pulling motion.

39. The method of claim 38, wherein the fiber is pulled at a rate of between about 0.1 mm/second and about 100 mm/second.

40. The method of claim 39, wherein the pulling force is generated by connecting the fiber to a circular winding means and rotating the winding means at the pull rate.

41. The method of claim 38, wherein the fiber comprises between two and 10,000 fibrelles.

42. The method of claim 41, wherein the fiber comprises between 10 and 1000 fibrelles.

43. The method of claim 24 wherein the fiber has a substantially homogenous diameter.

44. The method of claim 43, wherein the diameter is between about 0.1 μm and about 20 μm.

45. The method of claim 43, wherein the step of pulling the fiber from the interface is done at a rate less than the rate at which beading occurs.

46. The method of claim 24, wherein the fiber comprises domains of narrow diameter interposed with beads having a larger diameter.

47. The method of claim 46, wherein the step of pulling the fiber from the interface is done at a rate sufficient to induce beading.

48. The method of claim 46, wherein the bead has a diameter of between about 2 and about 25 times the diameter of the narrower domains.

49. A method of tissue engineering comprising the steps of:
(a) providing at least one fiber comprising at least two polyionic fiberelles and at least one bioactive material encapsulated within the fiber, or an article of manufacture composed of said fiber;
(b) shaping the fiber or article of manufacture into a two- or three-dimensional scaffold suitable for growth of the engineered tissue;
(c) contacting the scaffold with cells, growth factors, proteins, drugs, DNA, RNA, or combinations thereof under conditions conducive to tissue growth.

50. A product made by the process of claim 24.

51. The product of claim claim 50, wherein the bioactive material is selected from drugs, proteins, DNA, RNA, cells, viruses, microparticles, nanoparticles, contrast agents, or combinations thereof.

52. A method of treating a subject comprising administering a composition of claim 1 to the subject.

53. A method of generating tissue in a subject comprising administering a composition of claim 1 to the subject.

54. The fiber of claim 1, wherein each fibrelle is formed by interfacial polyelectrolyte complexation.

55. The fiber of claim 1, wherein each fiberelle is formed by interfacial complexation of at least one polycationic polymer and at least one polyanionic polymer.

56. The fiber of claim 1, wherein at least one polycationic polymer or at least one polyanionic polymer is biodegradable or biocompatible.
